(19) United States
(12) Patent Application Publication
(10) Pub. No.: US 2012/0029602 A1
(43) Pub. Date: Feb. 2, 2012

(54) Method, Apparatus, and System for Treatment of Hemorrhoidal Disease Using Negative Galvanism

(75) Inventors: Michael Knox, Tampa, FL (US); Michael Wodstrchill, New Port Richey, FL (US)

(73) Assignee: Vascular Technologies, Inc., St. Petersburg, FL (US)

(21) Appl. No.: 12/844,194

(22) Filed: Jul. 27, 2010

(51) Int. Cl. A61N 1/20

(52) U.S. Cl. 607/75

(57) ABSTRACT

Disclosed is a system to be used by a medical provider for treating hemmorhoidal tissue using negative galvanism. The system includes a single-use disposable medical probe, an ergonomically-shaped handle, a power supply, a display, a trigger, a plurality of lights, and a processing unit for activating the lights, and calculating and displaying the amount of electricity delivered to the hemmorhoidal tissue.

### BACKGROUND OF THE INVENTION

1. Field of the Invention

This invention relates to a method, apparatus and system for the treatment of Hemmorrhoidal Disease (HD) using negative galvanism.

2. Description of the Related Art

HD is currently treated with Infrared Coagulation (IRC), Rubber Band Ligation, Stapling and Surgery. By reference, Applicants incorporate the following patent publications into the present application: U.S. Pat. No. 4,898,169 to Norman et. al entitled Medical Instrument for Therapy of Hemorrhoidal Lesions; U.S. Patent Application Publication No. US 2006/0229595 to Newton et al entitled Instrument and Methods for Electrical Current Treatment of Tissues and Methods for Monitoring the Same; U.S. Patent Application Publication No. US 2008/0208188 to Cao entitled Probes for Electrical Current Therapy of Tissue, and Methods of Using Same; U.S. Patent Application Publication No. US 2009/0065565 to Cao entitled System, Method and Apparatus for Preventing Reuse of Medical Instruments. Applicants also incorporate by reference co-pending application U.S. Ser. No. 12/042,556 entitled Disposable Apparatus for Patient Grounding. Applicants also incorporate by reference the following: Canadian patent application no. 2597892 entitled Probes for Electrical Current Therapy of Tissue and Methods of Using Same; Costa Rican patent application no. 9321 entitled Probes for Electrical Therapy of Tissue and Methods of Using Same; European patent application no. 2006735114 entitled Probes for Electrical Current Therapy of Tissue and Methods of Using Same; Mexican patent application no. 2007009820 entitled Probes for Electrical Current Therapy of Tissue and Methods of Using Same.

Applicants also incorporate by reference the following: Dr. Daniel A. Norman, Direct Current Electrotherapy if Internal Hemorrhoids The American Journal of Gastroenterology, 1989.

It is an object of this invention to provide a more effective, safe, and reliable system, method and apparatus for the use of the galvanic method for the treatment of hemorrhoids.

It is a further object of this invention to provide a more effective system, method and apparatus for treating all grades (I-IV) of hemorrhoids.

It is a further object of this invention to improve patient safety in the treatment of HD.

It is a further object of this invention to improve disposability of the apparatus and system utilized in treating HD.

It is a further object of this invention to create an all-in-one system, method, apparatus and solution for treating HD which can be both portable and stationary.

It is a further object of this invention to create a researchable device to cut after-purchase costs for physicians.

The foregoing has outlined some of the pertinent objects of the invention. These objects should be construed to be merely illustrative of some of the more prominent features and applications of the intended invention. Many other beneficial results can be attained by applying the disclosed invention in a different manner or modifying the invention within the scope of the disclosure. Accordingly, other objects and a fuller understanding of the invention may be had by referring to the summary of the invention and the detailed description of the preferred embodiment in addition to the scope of the invention defined by the claims taken in conjunction with the accompanying drawings.

### SUMMARY OF THE INVENTION

For the purpose of summarizing this invention, this invention comprises a system for treating HD using negative galvanism. More particularly, the system applies a galvanic direct current to the mucosal tissue surface of symptomatic internal hemorrhoids for their elimination. It consists of a main electricity (AC-powered) generator which produces direct current in milliamperes (mA) passed via a patient pad, through the body, to a probe (an electrode) applied to the hemorrhoidal tissue, a hand piece, and electrical cables. The tissue is subjected to mild electrical current creating a biochemical reaction that interrupts the hemorrhoid vasculature and shrinks the hemorrhoids.

The foregoing has outlined rather broadly the more pertinent and important features of the present invention in order that the detailed description of the invention that follows may be better understood so that the present contribution to the art can be more fully appreciated. Additional features of the invention will be described hereinafter which form the subject of the claims of the invention. It should be appreciated by those skilled in the art that the conception and the specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims.

### BRIEF DESCRIPTION OF THE DRAWINGS

For a full understanding of the nature and objects of the invention, reference should be had to the following detailed description taken in connection with the accompanying drawings in which:

FIG. 1 is a front elevational view of the system of the present invention.

FIG. 2 is a side elevational view of the system of the present invention.

FIG. 3 is a rear elevational view of the system of the present invention.

FIG. 4 is a rear elevational view of the system of the present invention in an alternative embodiment

FIG. 5 is a side view showing one mechanism for connecting a disposable probe to the handle as described herein.

FIG. 6 is a block diagram illustrating one embodiment of a processing unit in accordance with the teachings of the present disclosure.

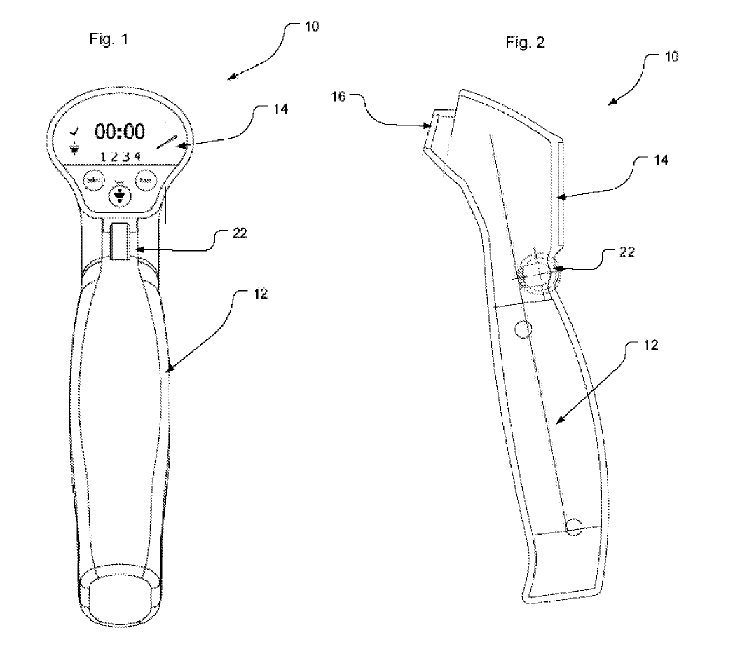

Similar reference characters refer to similar parts throughout the several views of the drawings.

### DETAILED DESCRIPTION OF THE PREFERRED EMBODIMENT

Reference is made to the attached figures and the following explanation for a description of the present invention

The present system 10 comprises the following components: Single-Use Probe 10, Handle 12, Base Unit (not depicted), Grounding Pad (not depicted), Single-Use Grounding Pad Sponge (not depicted), Single-Use Anoscope (not depicted), Grounding Pad Connection Cord (not depicted), and Power Cord (110, 220, etc.) (not depicted).

These components provide improved design and functionality to improve ergonomics as well as provide additional physician controls and safety test operations.

In a preferred embodiment, the Single-Use-Probe 18 interconnects with the Handle 12. Preferably, the handle 12 includes a socket 16 for receiving the single-use probe 18. The Handle 12 interconnects with the Base Unit. The Base Unit interconnects with the Grounding Pad sponge. Of course, as the skilled artisan will appreciate, other variations are possible. By using the term connection herein, it should be understood that it is not required that there be a part-to-part exchange.

The handle 12 comprises a screen or display 14 which is built around the new capabilities of device and future capabilities. The display 14 shows, inter alia, grounding test confirmation, the time of the procedure, and the milliamp level.

Turning to the figures, the system 10 disclosed herein is depicted. As depicted, the handle 12 is preferably ergonomically shaped so as to comfortably sit in the treating personnel s hand. Integrated into the handle 12 (and discussed below) are the display 14, a trigger 22 for controlling the amount of treatment provided, and a plurality of light sources 20. Additionally, the system 10 includes a probe chip 24 for calculating the amount of treatment provided to the tissue, activating the lights 20, and displaying the treatment time (and other information) to the medical personnel using the system 10.

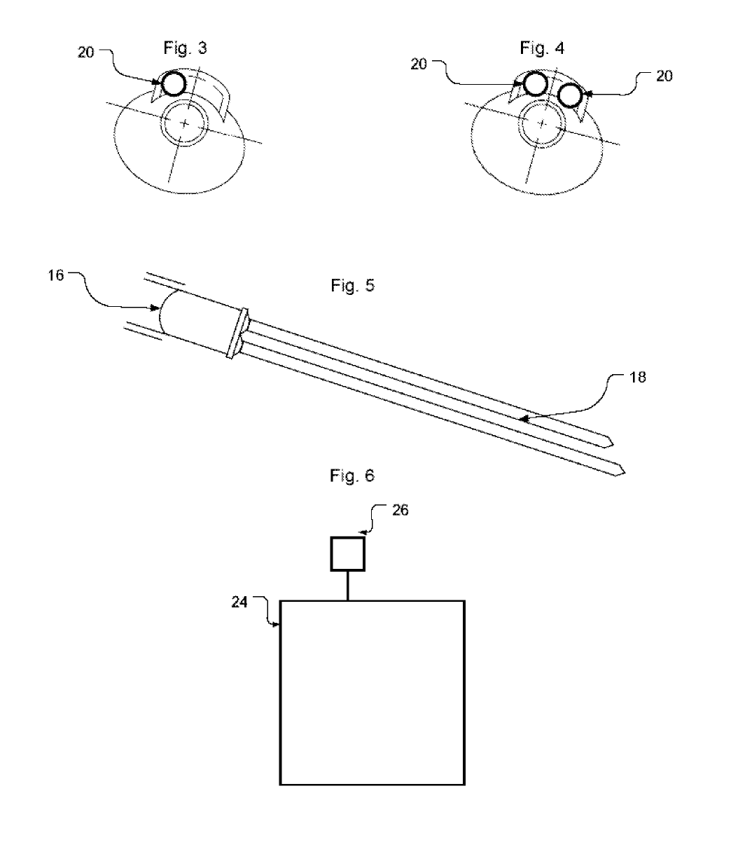

The handle 12 also can include a light source 20 to assist the user. The light source 20 is preferably implemented with two light-emitting diodes (LEDs) positioned just above where the single use probe 18 connects to the handle 12 (preferably through socket 16). See FIGS. 3 and 4. The light source 20 in the handle prevents the physician from needing a head lamp or a stationary lamp to use in conjunction with the procedure.

The system 10 also includes a probe chip 24, which can be a microprocessor or any other logic-type chip. The probe chip 24 may be able to assist in insuring that the probes are single use or at the least not used over 30 minutes (or any other predetermined time or current-time period) of consecutive treatment time. The probe chip 24 can be interconnected to a memory 26 capable of storing the steps necessary for the probe chip 24 to implement its features disclosed herein.

The integration of a light source 20, as the inventors have discovered, further enhances the system, method and apparatus by allowing the doctor or user to perform the treatment without a head lamp or standing lamp to go alongside each procedure.

The light 20 is achieved by integrating it with the unit circuitry and actually making it part of the handle 12 internal components.

The handle 12 can also include a scrolling mechanism to permit the user to increase and decrease the power being delivered. Alternatively, the trigger 22 can be a rheostatic trigger providing variable control of the amount of electricity provided through the probe 18.

The inventors have discovered that some of the many improvements herein are the installed light source, new molds, new milliamp control, battery permanently installed in the unit and the use negative galvanism applied to the base of a hemorrhoid, causing it to shrink and be reabsorbed into the body. The hemorrhoid does not reoccur in the location treated by the system, method and apparatus described herein.

The present disclosure includes that contained in the appended claims, as well as that of the foregoing description. Although this invention has been described in its preferred form with a certain degree of particularity, it is understood that the present disclosure of the preferred form has been made only by way of example and that numerous changes in the details of construction and the combination and arrangement of parts may be resorted to without departing from the spirit and scope of the invention.

Now that the invention has been described,

What is claimed is:

1. A system to be used by a medical provider for treating hemmorhoidal tissue using negative galvanism, the system comprising in combination:
a single-use disposable medical probe having at least two electrical conductors for delivering electricity to the hemmorhoidal tissue to be treated;
an ergonomically-shaped handle for removably accepting the disposable probe, the handle physically supporting the disposable probe;
a power supply associated with the handle, the handle being coupled to the two electrical conductors such that the power supply is in electrical contact with the conductors;
a display integrated into the handle for communicating to the medical provider an amount of treatment time, the display being angled with respect to the handle, whereby the display is readable by the medical provider when the handle is in a horizontal orientation;
a rheostatic trigger integrated into the handle and in electrical communication with the power supply, the trigger selectively activating the plurality of lights and the power supply such that the amount of electricity delivered to the tissue can be regulated;
a plurality of lights integrated into the handle opposite to the display for selectively illuminating the hemmorhoidal tissue being treated; and
a processing unit associated with the handle, whereby when the trigger activates the plurality of lights and the power supply, the amount of electricity delivered to the hemmorhoidal tissue can be determined by the processing unit and displayed on the display.
